(19) United States
(12) Patent Application Publication
(10) Pub. No.: US 2026/0314976 A1
(43) Pub. Date: Oct. 15, 2026

(54) EMPLOYING REFERENCE ELECTRODE POSITIONED ON SHEATH TO IMPROVE SIGNALS ACQUIRED BY CATHETER ELECTRODES

(71) Applicant: BIOSENSE WEBSTER (ISRAEL) LTD., Yokneam (IL)

(72) Inventors: Itai Doron, Katsir (IL); Meir Bar-Tal, Haifa (IL); Ido Ilan, Yoqneam (IL)

(21) Appl. No.: 19/179,263

(22) Filed: Apr. 15, 2025

(51) Int. Cl. A61B 5/287; A61B 5/00; A61B 5/367

(52) U.S. Cl. CPC A61B 5/287; A61B 5/367; A61B 5/7203

(57) ABSTRACT

A system for improving far-field noise cancellation from electrophysiological (EP) signals, includes: a catheter having catheter electrodes, a sheath configured to allow passage of the catheter having the catheter electrodes for collecting EP signals in an organ of a patient, the catheter passing through a distal end of the sheath, be navigated in the organ to a blood pool and avoid contact with tissue of the organ, the sheath includes at least a sheath electrode, coupled to the distal end of the sheath, and configured to acquire a reference signal indicative of a far-field signal generated by the organ, and a processor configured to use the reference signal for cancellation of the far-field noise in the EP signals acquired by at least one of the catheter electrodes, calculate distances between the sheath electrode and of the catheter electrodes, respectively, and normalize the EP signals based on the calculated distances.

### FIELD OF THE DISCLOSURE

The present disclosure relates generally to catheterization systems, and particularly to methods and systems for improving the quality of electrophysiological signals acquired by catheter electrodes using a reference electrode coupled to a sheath of a catheterization system.

### BACKGROUND OF THE DISCLOSURE

Various techniques for performing cardiac electro-anatomical mapping have been published.

For example, U.S. Pat. No. 11,540,878 describes a catheter shaft assembly including an outer sheath with a distal end, and an end effector associated with a distal end of the catheter shaft assembly that includes leaflets having a flexible body and a plurality of electrodes.

The present disclosure will be more fully understood from the following detailed description of the examples thereof, taken together with the drawings in which:

### BRIEF DESCRIPTION OF THE DRAWINGS

FIG. 1 is a schematic, pictorial illustration of a catheter-based system configured to carry out electrophysiological procedures, in accordance with an example of the present disclosure;

FIG. 2 is a schematic, pictorial illustration of a catheter and a sheath of the system of FIG. 1, in accordance with an example of the present disclosure; and

FIG. 3 is a flow chart that schematically illustrates a method for improving electro-anatomical mapping using reference electrode(s) mounted on the sheath of the system of FIG. 1, in accordance with an example of the present disclosure.

### DETAILED DESCRIPTION OF EXAMPLES

### Overview

The present disclosure addresses the problem of far-field noise interference in electro-anatomical (EA) mapping procedures of one or more regions in a heart by collecting electrophysiological (EP) signals during catheter-based procedures. Traditional reference electrodes on diagnostic catheters can inadvertently contact tissue, leading to errors in noise cancellation and signal interpretation.

Examples of the present disclosure that are described hereafter provide methods and system for improving the quality of EA mapping procedures by using a navigated sheath with one or more reference electrodes coupled to a distal end of the sheath, which is usually positioned within a blood pool of the heart, and thereby, avoiding tissue contact and providing a stable reference signal.

In some examples, a system for EA mapping comprises a catheter having electrodes configured to acquire EP signals in tissue of the heart. The catheter passes through a sheath navigated by a physician into the chamber in question in the heart. In some examples, the sheath comprises at least one reference electrode (in the present example two reference electrodes, also referred to herein as sheath electrodes) coupled to a distal end of the sheath. The sheath is configured to keep its distal end within the blood pool and avoid tissue contact, so as to provide a stable reference signal as described above.

In some examples, the system comprises a processor configured to receive reference signals from the sheath electrode, uses these signals as a reference for cancellation of far-field noise from the EP signals received from the catheter electrodes. The processor is further configured to (i) calculate the distance between each of the sheath electrodes and each of the catheter electrodes, and (ii) normalize the catheter signals based on the calculated distance. It is noted that by coupling the reference electrode(s) to the sheath, the reference signals remain stable and unaffected by movement of the catheter.

In some examples, the processor is configured to normalize the EP signals received from the catheter electrodes by considering the calculated distance, utilizing intensity decay rates of signal propagation in the blood pool. In some examples, the processor is configured to integrate the reference signals to refine the calculation of a Tissue Proximity Index (TPI) indicative of whether one or more of the catheter electrodes are touching the tissue of the chamber in question while collecting the EP signals. This approach considers, in the TPI calculation, not only the characteristics of impedance readings received from the electrodes, but also those of the ECG signals of the reference signals received from the sheath electrode(s). This technique enhances the traditional impedance-focused TPI calculations by incorporating electrocardiogram (ECG) signal data from the reference electrode, and thereby, improves the quality of EP signal acquisition. Moreover, using reference electrodes mounted on the sheath rather than on the catheter allows for (i) the use of larger-size reference electrodes (compared to reference electrodes mounted on the catheter's distal portion), and (ii) obtaining stable reference signals that are not affected by movement of the catheter within the patient heart.

### System Description

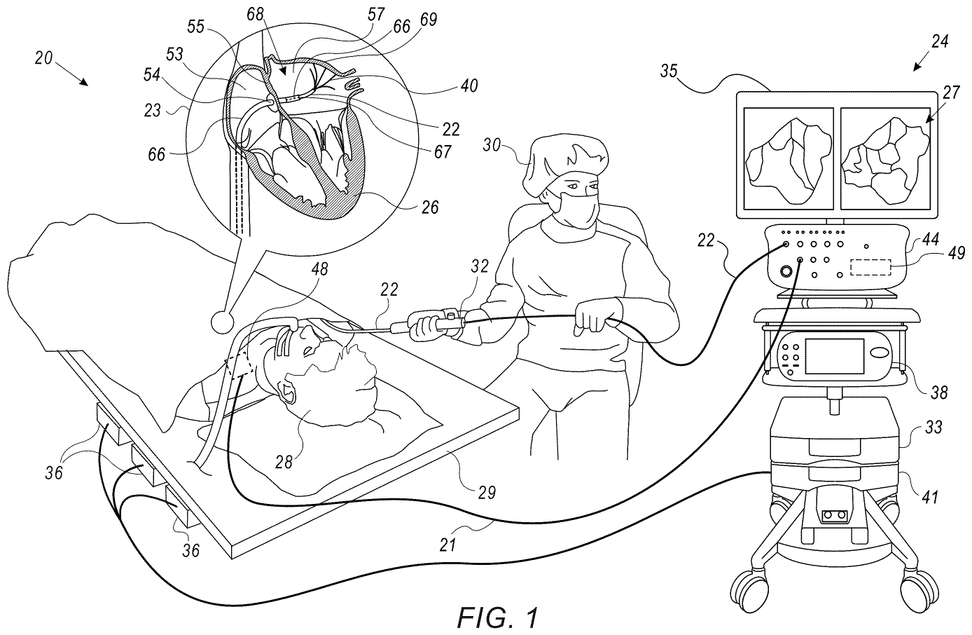

FIG. 1 is a schematic, pictorial illustration of a catheter-based system 20 configured to carry out electrophysiological (EP) procedures in an organ of a patient 28, in accordance with an example of the present disclosure.

In some examples, system 20 comprises a catheter 22 and a control console 24, both described in detail below. In the present example, catheter 22 is configured to collect EP signals (e.g., electro-anatomical signals) in the organ (e.g., a heart 26 as described below). Additionally, or alternatively, catheter 22 may be used for any suitable therapeutic and/or diagnostic purposes, such as ablation of tissue in heart 26.

In some examples, console 24 comprises a processor 33, typically a general-purpose computer, with suitable front end and interface circuits for receiving signals via catheter 22 and for controlling the other components of system 20 described herein. Console 24 further comprises a user display 35, which is configured to receive from processor 33 graphical and/or textual display items, such as a map 27 of heart 26, and to display map 27.

In some examples, map 27 may comprise any suitable type of three-dimensional (3D) anatomical map produced using any suitable technique. For example, the anatomical map may be produced using an anatomical image produced by using a suitable medical imaging system, or using a fast anatomical mapping (FAM) technique available in the CARTO™ system, produced by Biosense Webster Inc. (Irvine, Calif.), or using any other suitable technique, or using any suitable combination of the above.

In some examples, console 24 comprises a recording unit 38, which is configured to record in case of failure in the CARTO™ and/or a failure to pace in certain electrodes. Console 24 comprises a patient interface unit (PIU) 44, which is configured to produce a signal indicative of the location and electrocardiogram (ECG) signals that are acquired and processed and to exchange signals between console 24 and multiple entities (e.g., catheter 22) of system 20.

Reference is now made to an inset 23. In some examples, prior to performing an ablation procedure, a physician 30 inserts one or more catheters 22 through the vasculature system of patient 28 lying on a table 29, so as to perform electro-anatomical (EA) mapping of tissue in question of heart 26. Based on the EA mapping, physician 30 plans the RF ablation or IRE procedure.

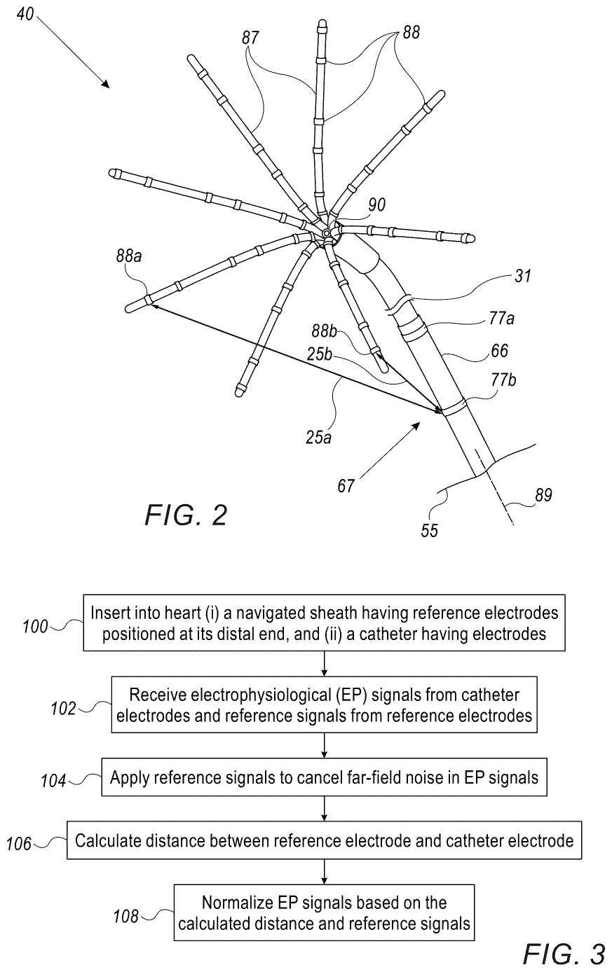

In some examples, system 20 comprises a sheath 66 configured to allow passage of catheter 22 having one or more electrodes (shown in FIG. 2 below) for collecting the EP signals.

In some examples, catheter 22 comprises a distal-end assembly 40 having multiple electrodes disposed on one or more surfaces of distal-end assembly 40. An example implementation of distal end assembly 40 is shown and described in detail in FIG. 2 below. In the present example, physician 30 intends to perform the aforementioned EA mapping procedure at intended locations on the surface of a left atrium 57 of heart 26. Note that for clinical considerations, the procedure requires: (i) insertion and navigation of sheath 66, through the vasculature system of patient 28, into a right atrium 53 of heart 26, (ii) puncturing a hole 54 (also referred to herein as a transseptal hole) in a septum 55 between right atrium 53 and left atrium 57, (iii) threading sheath 66 through hole 54 into the intended location in left atrium 57, (iv) moving distal end assembly 40 of catheter 22 through a distal end 67 of sheath 66 into the intended location of tissue 69 in left atrium 57, and (v) performing the electro-anatomical (EA) mapping procedure by placing the electrodes of distal end assembly 40 in contact with the intended tissue 69 and collecting EP signals from the tissue.

The present example describes an EA mapping carried out in left atrium 57 of heart 26 by puncturing hole 54 in septum 55. It is noted that the disclosed techniques are applicable, mutatis mutandis, to any EA mapping carried out at any other location in heart 26, or in any other suitable organ of patient 28.

In the present example, distal end 67 of sheath 66 is configured to be navigated to a blood pool 68 in left atrium 57 and avoid contact with tissue 69. In some examples, the puncturing of hole 54 may be carried out using a guidewire (not shown) or any other guidewire of catheter 22 or of another catheter inserted into right atrium 53.

Reference is now made back to the general view of FIG. 1. In some examples, the proximal end of catheter 22 is connected, inter alia, to interface circuits (not shown) of PIU 44, for transferring electrocardiogram (ECG) signals collected by the electrodes of catheters 22 (shown in FIG. 2 below) from tissue of heart 26, and position signals (described below) to processor 33 for performing the EA mapping.

In some examples, PIU 44 is connected to a power source, such as a radiofrequency (RF) generator 49, which is packaged within the housing of PIU 44 and is configured to apply ablation signals during ablation procedures. In alternative examples, the RF generator may be external to the housing of PIU 44 and electrically connected to PIU 44 using a suitable cable.

In some examples, RF generator 49 is configured to apply RF signals having a voltage larger than about 1000 V, in the present example, between about 1200 V and 2000 V. In some examples, system 20 comprises an indifferent electrode 48, also referred to herein as a patch electrode, which is attached to the skin of patient 28 (e.g., on the backside of patient torso) and is electrically connected to PIU 44 via a cable 21.

In some examples, system 20 is configured to puncture the tissue of septum 55 using electrical induced by applying one or more unipolar pulsed irreversible electroporation (IRE) signals, having about 1200 V or 1800 V or 2000 V (or any other suitable voltage), between the aforementioned guidewire and indifferent electrode 48.

In the context of the present disclosure and in the claims, the terms “about” or “approximately” for any numerical values or ranges indicate a suitable dimensional tolerance that allows the part or collection of components to function for its intended purpose as described herein.

In some examples, after forming hole 54 physician 30 inserts sheath 66 through hole 54 so that distal end 67 of sheath is positioned within blood pool 68 of left atrium 57. Moreover, physician 30 navigates distal-end assembly 40 into the target location in tissue 69 of left atrium 57 for placing the electrodes of catheter 22 in contact with the target location, so as to collect the EP signals from tissue 69.

In some examples, system 20 comprises a handle 32 used by physician 30 to: (i) navigate sheath 66 into right atrium 53, (ii) produce hole 54 in septum 55, (iii) navigate sheath 66 through hole 54 and further navigate distal end assembly 40 to the target location for performing the EA mapping procedure in left atrium 57.

In some examples, the position of distal-end assemblies 40 and 67 in the vasculature and heart 26 of patient 28 are measured using a position sensor (not shown) of a magnetic position tracking system. In the present example, console 24 comprises a driver circuit 41, which is configured to drive magnetic field generators 36 placed at known positions external to patient 28 lying on table 29, e.g., below the patient's torso. The position sensor is coupled to at least one of the distal ends 40 and 67 and is configured to generate position signals in response to sensed external magnetic fields from field generators 36. The position signals are indicative of the position of the respective distal end of sheath 66 and/or catheter 22 in the coordinate system of the position tracking system.

This method of position sensing is implemented in various medical applications, for example, in the CARTO^{TM} system, produced by Biosense Webster Inc. (Irvine, Calif.) and is described in detail in U.S. Pat. Nos. 5,391,199, 6,690,963, 6,484,118, 6,239,724, 6,618,612 and 6,332,089, in PCT Patent Publication WO 96/05768, and in U.S. Patent Application Publications 2002/0065455 A1, 2003/0120150 A1 and 2004/0068178 A1.

In some examples, the coordinate system of the position tracking system is registered with the coordinate systems of system 20 and map 27, so that processor 33 is configured to display, the position of distal-end assembly 40, over the anatomical or EA map (e.g., map 27).

In some examples, processor 33, typically comprises a general-purpose computer, which is programmed in software to carry out the functions described herein. The software may be downloaded to the computer in electronic form, over a network, for example, or it may, alternatively or additionally, be provided and/or stored on non-transitory tangible media, such as magnetic, optical, or electronic memory.

This particular configuration of system 20 is shown by way of example, in order to illustrate certain problems that are addressed by examples of the present disclosure and to demonstrate the application of these examples in enhancing the performance of such a system. Examples of the present disclosure, however, are by no means limited to this specific sort of example system, and the principles described herein may similarly be applied to other sorts of medical systems.

### Employing Sheath Reference Electrode to Improve Catheter electrodes signals

FIG. 2 is a schematic, pictorial illustration of distal end assembly 40 of catheter 22 and distal end 67 of sheath 66, in accordance with an example of the present disclosure.

In some examples, sheath 66 is configured to allow the passage of distal end assembly 40, which is equipped with one or more electrodes 88 for collecting EP signals at the target location in tissue 69 (shown in FIG. 1 above) of heart 26.

In some examples, sheath 66 comprises one or more (e.g., two) sheath electrodes also referred to herein as reference electrodes 77a and 77b. In the present example, reference electrodes 77a and 77b are: (a) coupled to distal end 67 of sheath 66, and (b) configured to produce reference signals indicative of far-field signals generated by portions of or areas within tissue 69, located farther away from the catheter 40. It is noted that the length of the shaft of catheter 22 being moved out of distal end 67 of sheath 66 is longer than that shown in FIG. 2, as illustrated by a continuity symbol 31 of FIG. 2 and in the schematic illustration of FIG. 1 above.

In some examples, distal end 67 and reference electrodes 77a and 77b are configured to remain within the blood pool 68 (shown in FIG. 1 above) and avoid contact with tissue 69. Typically, distal end 67 of sheath 66 remains in the blood pool without being actively moved (or with minor movements), thereby reference electrodes 77a and 77b are configured to provide reliable reference signals indicative of the far-field signal generated by heart 26.

In some examples, processor 33 is configured to use the reference signals to cancel far-field noise in the EP signals collected by the electrodes 88.

In some examples, electrodes 88 are distributed along distal end assembly 40 of catheter 22. In the present example, catheter 22 comprises a WEBSTER™ Octopolar deflectable catheter, OCTARAY™ supplied by Biosense Webster Inc. and designed to facilitate electrophysiological mapping of selected regions in heart 26. In this configuration distal end assembly 40 comprises eight arms 87 (also referred to herein as splines), each of which comprising six electrodes 88, so that in total distal end assembly 40 comprises 48 electrodes 88 configured to collect the EP signals from tissue 69.

In other examples, catheter 22 may comprise any other suitable type of distal end assembly, such as but not limited to a PENTARAY® NAV ECO High Density Mapping Catheter supplied by Biosense Webster Inc. and having five arms with electrodes for collecting the EP signals.

In some examples, in addition to the cancellation of the far-field noise in the EP signals, processor 33 is configured to calculate distances between (i) each of the reference electrodes 77a and 77b, and (ii) each of the electrodes 88, and normalizes the EP signals based on these calculated distances. For example, electrodes 88a and 88b are located at respective distances 25a and 25b from reference electrode 77b. As such, processor 33 is configured to normalize the EP signals collected by electrodes 88a and 88b using different factors determined, inter alia, by distances 25a and 25b.

In some examples, reference electrodes 77a and 77b are positioned at different locations along an axis 89 indicative of the length of sheath 66. In some examples, processor 33 is configured to select the reference electrode 77 with the most distance value from the nearest tissue (e.g., tissue 69) for use as the reference signal. In the present example, reference electrode 77b is positioned along axis 89 proximally relative to reference electrode 77a, thus, farther than tissue 69 compared to reference electrode 77a. This selection ensures that the reference signal is not affected by the movement of distal end assembly 40 of catheter 22 within heart 26 and improves the quality and reliability of the far-field noise cancellation from the EP signals collected by catheter electrodes 88. In other examples, the processor 33 is configured to select the reference electrode 77 according to other considerations or readings, for example, contact indication, ECG signals, ECG morphology, chamber location, and the like.

In some examples, processor 33 is configured to calculate a Tissue Proximity Index (TPI), which is indicative of whether electrodes 88 are touching the tissue in question while collecting the EP signals. By integrating reference signals from the sheath-mounted reference electrodes 77a and 77b, processor 33 is configured to refine and improve the estimation accuracy of the TPI. In some examples, this approach considers not only the characteristics of the impedance readings measured by electrodes 88, but also the readings of the ECG signals acquired by at least one of reference electrodes 77a and 77b. This technique enhances the traditional impedance-focused TPI calculations by incorporating ECG signal data from at least one of the reference electrodes 77a and 77b. Moreover, in the context of the present disclosure and in the claims, the terms “touch calibration” and “TPI enhancement” and grammatical variations thereof, are used interchangeably and refer to improvements in the accuracy of an estimation of the Tissue Proximity Index (TPI).

In some cases, catheters may comprise a reference electrode mounted on the distal end assembly, for example, in the aforementioned Octopolar deflectable catheter a reference electrode may be positioned at a position 90. This reference electrode is typically smaller than electrodes 88 and being moved together with distal end assembly 40 while physician 30 navigates catheter 22, so that quality and stability of the reference signal might be insufficient. In some examples, using reference electrodes coupled to sheath 66, such as electrodes 77a and 77b, allows for the use of larger-size reference electrodes coupled. For example, reference electrode 77 may have a size that matches at least half of the size of electrodes 88 and typically having the same size or even a larger size compared to that of at least one of and typically all of electrodes 88.

In some examples, processor 33 is configured to extract electrocardiogram (ECG) data from the reference signals received from at least one of reference electrodes 77a and 77b, and based on the ECG data, processor 33 is configured to improve the accuracy of the EP signals received from electrodes 88.

In some examples, the use of one or more sheath electrodes, such as reference electrodes 77a and 77b (in the example of FIG. 2) for signal analysis provides significant advantages that are applicable across various catheter types and designs. This approach improves the quality and reliability of electrophysiological signal analysis regardless of the specific configuration of catheter 22 (or any other catheter used in such procedures) or the presence or absence of reference electrodes on catheter 22 itself.

The disclosed technique associated with sheath electrode(s) 77 may be employed with any type of catheter (e.g., catheter 22), such as but not limited to mapping catheters, ablation catheters, diagnostic catheters, and therapeutic catheters. This versatility stems from the fact that at least one of the sheath electrodes 77 remains substantially stationary in the blood pool 68, thereby providing a stable reference point for signal analysis that is independent of movement and/or the design of the utilized catheter (e.g., catheter 22).

Moreover, utilizing sheath electrodes 77 for reference signals eliminates the need to incorporate dedicated reference electrodes into the design of catheter 22. This may simplify the design and fabrication of catheter 22, and may reduce manufacturing costs and complexity. It may also allow for more flexible catheter 22 designs, as the space that would otherwise be occupied by a catheter-mounted reference electrode can be utilized for other purposes or left free to create a more compact catheter profile.

The sheath electrode 77 approach may also provide more consistent and reliable reference signals across different procedures and catheter types. Since the positioning of sheath 66 tends to remain more stable throughout a procedure compared to the (typically) constantly moving distal end assembly 40, the reference signal acquired from the sheath electrode 77 may be less susceptible to motion artifacts and local tissue effects. This stability may contribute to improved accuracy in noise cancellation, signal normalization, and other signal processing techniques across a wide range of electrophysiological procedures.

It is noted that the disclosed techniques enhance the quality of electrograms (e.g., intracardiac electrograms), facilitate TPI enhancement, and improve the overall effectiveness of the EP mapping procedure carried out in heart 26. The disclosed techniques are applicable, mutatis mutandis, in any other suitable catheter type and/or organ of patient 28.

FIG. 3 is a flow chart that schematically illustrates a method for improving electro-anatomical mapping in heart 26 using reference electrodes 77a and 77b mounted on sheath 66, in accordance with examples of the present disclosure.

The method begins at a sheath and catheter insertion step 100 with inserting into heart 26 (i) sheath 66 having electrodes 77a and 77b, and (ii) distal end assembly 40 (of catheter 22) having electrodes 88, as described in detail in FIGS. 1 and 2 above.

At a signal receiving step 102, processor 33 receives EP signals from electrodes 88 and reference signals from electrodes 77a and 77b, as described in detail in FIG. 2 above. At a noise cancellation step 104, processor 33 applies the reference signals to cancel far-field noise in EP signals, as described in detail in FIG. 2 above. At a distance calculating step 106, processor 33 calculates distances between each reference electrode 77 and each electrode 88. For example, processor 33 calculates distance 25a between electrodes 77b and 88a, and distance 25b between electrodes 77b and 88b, as described in detail in FIG. 2 above. At a signal normalizing step 108 that concludes the method, processor normalizes the EP signals received from electrodes 88 (e.g. from electrodes 88a and 88b) based on (i) the calculated distance obtained in step 106 above, and (ii) the reference signals received from reference electrodes 77a and 77b, as described in detail in FIG. 2 above.

In some examples, processor 33 is further configured to improve the accuracy of an estimation of the Tissue Proximity Index (TPI), which is indicative of whether a given electrode 88 is placed in contact with the tissue in question (e.g., tissue 69). Processor 33 is further configured to extract electrocardiogram (ECG) data from the reference signal, and to improve the accuracy of the EP signals based on the ECG data, as described in FIG. 2 above. Moreover, in some examples, electrodes 77a and 77b are positioned within blood pool 68 along with distal end 67 of sheath 66 that typically does not move during the EA mapping procedure. In such examples, electrodes 77a and 77b are configured to produce the reference signals, which are typically not affected by the movement of distal end assembly 40 within left atrium 57 and thereby improve the stability of the reference signals and improve the far-field noise cancellation in the EP signals acquired by electrodes 88, as described in FIG. 2 above. In other words, sheath 66 is configured to maintain the position of the one or more reference electrodes 77 substantially stationary. More specifically, during a medical procedure (e.g., electrophysiological mapping) the reference electrode(s) 77 generally remains stationary or move significantly less within the blood pool 68 of heart 26 compared to the movements of electrodes 88 of distal end assembly 40. Consequently, at least one of reference electrodes 77 is configured to acquire the reference signal that is less prone to motion artifacts than the signals from electrodes 88 of catheter 22. Additionally, the stationary nature of the reference electrodes 77 allows for significant movement of electrodes 88 and the collection of electrophysiological (EP) signals by electrodes 88 without introducing noise related to the motion of distal end assembly 40.

Although the examples described herein mainly address reducing far-field noise in EP signals acquired in a patient's heart, the methods and systems described herein may also be considered for use in other applications.

### EXAMPLE 1

A system (20) for improving far field noise cancellation from electrophysiological (EP) signals, the system includes: (i) a catheter (22), including one or more catheter electrodes (88, 88a, 88b), (ii) a sheath (66) configured to (a) allow passage of the catheter having the one or more catheter electrodes for collecting EP signals in an organ (26) of a patient (28), the catheter passing through a distal end of the sheath, (b) be navigated in the organ to a blood pool (68) and avoid contact with tissue (69) of the organ, the sheath includes at least a sheath electrode (77a, 77b), which is: (i) coupled to the distal end of the sheath, and (ii) configured to acquire a reference signal indicative of a far-field signal generated by the organ, and (iii) a processor (33) configured to: (a) use the reference signal for cancellation of the far-field noise in the EP signals acquired by at least one of the one or more catheter electrodes, (b) calculate one or more distances (25a, 25b) between (i) the sheath electrode and (ii) one or more of the catheter electrodes, respectively, and (c) normalize the EP signals based on the one or more calculated distances.

### EXAMPLE 2

The system according to Example 1, wherein, based the reference signal, the processor is configured to improve accuracy of an estimation of a Tissue Proximity Index (TPI), indicative of whether a given electrode among the one or more catheter electrodes is placed in contact with the tissue.

### EXAMPLE 3

The system according to Example 2, wherein the processor is configured to improve the accuracy of the TPI based on a first position of the sheath electrode relative to one or more second positions of the one or more catheter electrodes.

### EXAMPLE 4

The system according to Example 1, wherein the processor is configured to normalize the EP signals by considering the calculated distance between the sheath electrode and the catheter electrodes, utilizing intensity decay rates of signal propagation in the blood pool modeled as inversely proportional to the calculated distance.

### EXAMPLE 5

The system according to Example 1, wherein the sheath includes first and second sheath electrodes, positioned at first and second distances from the tissue, respectively, the first and second sheath electrodes being configured to produce first and second reference signals, respectively, wherein the second distance is larger than the first distance, and wherein, based on the first and second distances, the processor is configured to select the second reference signal for cancellation of the far-field noise in the EP signals.

### EXAMPLE 6

The system according to Example 1, wherein the processor is configured to extract electrocardiogram (ECG) data from the reference signal, and to improve accuracy of the EP signals based on the ECG data.

### EXAMPLE 7

The system according to Example 1, wherein the sheath electrode has a first size that matches at least half of a second size of at least one of the catheter electrodes.

### EXAMPLE 8

The system according to Example 1, wherein the sheath is configured to: (i) pass through a septum of the organ, and (ii) be positioned within the blood pool, and wherein the sheath electrode is configured to produce the reference signal, which is not affected by a movement of the catheter in the organ.

### EXAMPLE 9

The system according to Example 1, wherein the sheath is configured to (i) maintain a position of the sheath electrode within the blood pool of the organ, and (ii) allow movement of the catheter, and wherein the sheath electrode is configured to acquire the reference signal that is less susceptible to motion artifacts compared to EP signals collected by the catheter electrodes.

### EXAMPLE 10

The system according to Example 1, wherein the organ includes a heart.

### EXAMPLE 11

A method for improving far field noise cancellation from electrophysiological (EP) signals, the method includes (a) inserting, through a sheath (66) into an organ (26) of a patient (28), a catheter (22) including one or more catheter electrodes (88, 88a, 88b), wherein the sheath includes at least a sheath electrode (77a, 77b) coupled to a distal end of the sheath, (b) the sheath is navigated in the organ to a blood pool (68) while avoiding contact with tissue (69) of the organ, (c) EP signals are collected using the one or more catheter electrodes, a reference signal indicative of a far-field signal generated by the organ using the sheath electrode is acquired, and using the reference signal for cancellation of the far-field noise in the EP signals acquired by at least one of the one or more catheter electrodes, (d) one or more distances (25a, 25b) between (i) the sheath electrode and (ii) one or more of the catheter electrodes, respectively, are calculated, and (e) the EP signals are normalized based on the one or more calculated distances.

It will be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A system for improving far-field noise cancellation from electrophysiological (EP) signals, the system comprising:
a catheter, comprising one or more catheter electrodes;
a sheath configured to (i) allow passage of the catheter having the one or more catheter electrodes for collecting EP signals in an organ of a patient, the catheter passing through a distal end of the sheath, (ii) be navigated in the organ to a blood pool and avoid contact with tissue of the organ, the sheath comprises at least a sheath electrode, which is: (a) coupled to the distal end of the sheath, and (b) configured to acquire a reference signal indicative of a far-field signal generated by the organ; and
a processor configured to:
use the reference signal for cancellation of the far-field noise in the EP signals acquired by at least one of the one or more catheter electrodes;
calculate one or more distances between (i) the sheath electrode and (ii) one or more of the catheter electrodes, respectively; and
normalize the EP signals based on the one or more calculated distances.

2. The system according to claim 1, wherein, based the reference signal, the processor is configured to improve accuracy of an estimation of a Tissue Proximity Index (TPI), indicative of whether a given electrode among the one or more catheter electrodes is placed in contact with the tissue.

3. The system according to claim 2, wherein the processor is configured to improve the accuracy of the TPI based on a first position of the sheath electrode relative to one or more second positions of the one or more catheter electrodes.

4. The system according to claim 1, wherein the processor is configured to normalize the EP signals by considering the calculated distance between the sheath electrode and the catheter electrodes, utilizing intensity decay rates of signal propagation in the blood pool modeled as inversely proportional to the calculated distance.

5. The system according to claim 1, wherein the sheath comprises first and second sheath electrodes, positioned at first and second distances from the tissue, respectively, the first and second sheath electrodes being configured to produce first and second reference signals, respectively, wherein the second distance is larger than the first distance, and wherein, based on the first and second distances, the processor is configured to select the second reference signal for cancellation of the far-field noise in the EP signals.

6. The system according to claim 1, wherein the processor is configured to extract electrocardiogram (ECG) data from the reference signal, and to improve accuracy of the EP signals based on the ECG data.

7. The system according to claim 1, wherein the sheath electrode has a first size that matches at least half of a second size of at least one of the catheter electrodes.

8. The system according to claim 1, wherein the sheath is configured to: (i) pass through a septum of the organ, and (ii) be positioned within the blood pool, and wherein the sheath electrode is configured to produce the reference signal, which is not affected by a movement of the catheter in the organ.

9. The system according to claim 1, wherein the sheath is configured to (i) maintain a position of the sheath electrode within the blood pool of the organ, and (ii) allow movement of the catheter, and wherein the sheath electrode is configured to acquire the reference signal that is less susceptible to motion artifacts compared to EP signals collected by the catheter electrodes.

10. The system according to claim 1, wherein the organ comprises a heart.

11. A method for improving far-field noise cancellation from electrophysiological (EP) signals, the method comprising:
inserting, through a sheath into an organ of a patient, a catheter comprising one or more catheter electrodes, wherein the sheath comprises at least a sheath electrode coupled to a distal end of the sheath;
navigating the sheath in the organ to a blood pool while avoiding contact with tissue of the organ;
collecting EP signals using the one or more catheter electrodes, acquiring a reference signal indicative of a far-field signal generated by the organ using the sheath electrode, and using the reference signal for cancellation of the far-field noise in the EP signals acquired by at least one of the one or more catheter electrodes;
calculating one or more distances between (i) the sheath electrode and (ii) one or more of the catheter electrodes, respectively; and
normalizing the EP signals based on the one or more calculated distances.

12. The method according to claim 11, further comprising improving, based on the reference signal, accuracy of an estimation of a Tissue Proximity Index (TPI), indicative of whether a given electrode among the one or more catheter electrodes is placed in contact with the tissue.

13. The method according to claim 12, wherein improving the accuracy of the TPI is based on a first position of the sheath electrode relative to one or more second positions of the one or more catheter electrodes.

14. The method according to claim 11, wherein normalizing the EP signals comprises considering the calculated distance between the sheath electrode and the one or more catheter electrodes, utilizing intensity decay rates of signal propagation in the blood pool modeled as inversely proportional to the calculated distance.

15. The method according to claim 11, wherein the sheath comprises first and second sheath electrodes, positioned at first and second distances from the tissue, respectively, the first and second sheath electrodes and being configured to produce first and second reference signals, respectively, wherein the second distance is larger than the first distance, and wherein the method further comprises selecting, based on the first and second distances, the second reference signal for cancellation of the far-field noise in the EP signals.

16. The method according to claim 11, further comprising extracting electrocardiogram (ECG) data from the reference signal and improving accuracy of the EP signals based on the ECG data.

17. The method according to claim 11, wherein the sheath electrode has a first size that matches at least half of a second size of at least one of the catheter electrodes.

18. The method according to claim 11, further comprising (a) passing the sheath through a septum of the organ, (b) positioning the sheath within the blood pool, and (c) producing the reference signal, which is not affected by a movement of the catheter in the organ, using the sheath electrode.

19. The method according to claim 11, further comprising (a) maintaining a position of the sheath electrode within the blood pool of the organ, (b) allowing movement of the catheter, and (c) acquiring the reference signal using the sheath electrode, wherein the reference signal is less susceptible to motion artifacts compared to EP signals collected by the catheter electrodes.

20. The method according to claim 11, wherein the organ comprises a heart of the patient.
